(19) United States
(12) Patent Application Publication
(10) Pub. No.: US 2007/0148219 A1
(43) Pub. Date: Jun. 28, 2007

(54) Liposomal Nanowater-Containing Patch-Type Nanodermal Gel for Transdermal Delivery and Method for Preparing the Same

(75) Inventors: Jong Taek Jeon, Sokcho-si (KR); Young Ju Kim, Gangneung-si (KR); Ju Yong Kim, Sokcho-si (KR)

Correspondence Address: STEPTOE & JOHNSON LLP, 1330 CONNECTICUT AVENUE, N.W., WASHINGTON, DC, 20036, US

(73) Assignee: Sphere Tech Co., LTD., Yangyang-gun (KR)

(21) Appl. No.: 11/425,260

(22) Filed: Jun. 20, 2006

(30) Foreign Application Priority Data
Dec. 28, 2005 (KR) 10-2005-0131131

(51) Int. Cl. A61K 9/70

(52) U.S. Cl. 424/449

(57) ABSTRACT

Disclosed herein is a patch-type nanodermal gel including liposomal nanowater, which can deliver cosmetically active substances, such as those functioning to moisturize the skin, into and through the skin stably and effectively. The patch-type cosmetic composition for transdermal deliver, based on nanodermal gel, comprises nanowater in an amount from 0.01 to 80.0% by weight; a skin-compatible polymer in an amount from 0.01 to 10.0% by weight; and a polyhydric alcohol in an amount from 5.0 to 20.0% by weight, based on the total weight of the nanodermal gel.

### CROSS REFERENCE

This application claims priority under 35 U.S.C. 119(e) to Korean Patent Application No. 10-2005-0131131, filed on Dec. 28, 2005, the entire contents of which is hereby incorporated by reference.

### BACKGROUND OF THE INVENTION

1. Field of the Invention

The present invention relates to a patch-type nanodermal gel containing liposomal nanowater suitable for transdermal delivery, which is highly adhesive to the skin at the temperature of the body and delivers a cosmetically active substance contained in the nanowater into and through the skin effectively and stably. The present invention also pertains to a method for preparing the liposomal nanowater-containing patch-type nanodermal gel for transdermal delivery.

2. Description of the Related Art

Representative of transdermal delivery systems, hydrogel is known as a base carrier suitable for the controlled release of pharmaceutically active substances. Particularly, hydrogel is used as a drug carrier that is effective for hormone control, anti-inflammation, pain alleviation, and the treatment of rheumatic arthritis. In addition, hydrogel has attracted intensive attention in the cosmetic field due to its uses as a patch type cosmetic. When applied to the skin, hydrogel, when combined with active substances, is able to deliver them into and through the skin. However, not only does it take a lot of time to achieve transdermal delivery with hydrogel, but also only a limited amount of the active substances is absorb through the skin. In addition, local transdermal delivery occurs only at the region to which the patch is applied.

Extensive research has been conducted o overcome the limits of hydrogel. For instance, gel compositions based on highly absorptive polymers and hydrophilic polymers, such as acrylate polymers, mucopolysaccharides, etc, have been suggested as being able to stably contain active substances therein. It has been disclosed that hydrogel containing skin penetration enhancers can effectively release the active substances and show high transdermal activity. High adhesive hydrogel has also been disclosed. In addition, a multilayer structure has been applied to hydrogel in order to stabilize and controllably release the active substance (Korean Pat. Laid-Open Publication Nos. 2000-0061633, 1990-0002847, and 1990-004093 and Korean Pat, No. 10-0452372). Recently, hydrogel capable of changing phases with temperature has been developed (Korean Pat. No. 10-0506543). These hydrogels are found to have improved release control, transdermal delivery function, and skin adhesion, to an extent that is nevertheless unsatisfactory.

Hydrogel for use in the cosmetic art usually contains active ingredients which have functions of skin whitening, wrinkle prevention, moisturization, elasticity promotion, softening, etc. Various factors, including gel compositions, physical and chemical properties of the active substances, etc., limit the application and uses of the hydrogel. Also, although extensively studied by many researchers, the stability of active substances within the gel and upon release into the skin remains a problem to be solved.

### SUMMARY OF THE INVENTION

Accordingly, the present invention has been made keeping in mind the above problems occurring in the prior art, and an object of the present invention is to provide a patch-type cosmetic for transdermal delivery, which can retain a cosmetically active substance therein stably for a long period of time and deliver it into and through the skin within a short period of time effectively and stably.

It is another object of the present invention to provide a method for preparing the patch-type cosmetic.

In accordance with an aspect of the present invention, provided is a patch-type cosmetic composition for transdermal delivery, based on nanodermal gel, comprising: nanowater in an amount from 0.01 to 80.0% by weight; a skin-compatible polymer in an amount from 0.01 to 10.0% by weight; and a polyhydric alcohol in an amount from 5.0 to 20.0% by weight, based on the total weight of the nanodermal gel.

In accordance with another aspect of the present invention, provided is a method for preparing a patch-type cosmetic, comprising: uniformly dissolving a skin-compatible polymeric substance in purified water and a polyhydric alcohol at a warm temperature; homogeneously mixing the solution with nanowater at 40 to 50° C., along with a flavor and a colorant, by stirring to produce nanodermal gel including the nanowater therein; layering nanodermal gel to a thickness from 500 to 1,500 μm on a film; and cutting the film to a predetermined size for patching.

In an embodiment, the nanowater ranges in size from 20 to 100 nm and is prepared by mixing 2.0˜4.0 wt % of lecithin, 2.0˜5.0 wt % of cholesterol and 1.0˜2.5 wt % of beta-sitosterol based on the total weight of the nanowater, at 40˜50° C. by gently stirring at 60˜100 rpm using a paddle mixer and vigorously stirring at 2,500˜3,500 rpm using a homo mixer for 2 to 4 min, and then by passing the mixture at 700˜1,000 bar three times through a microfluidizer.

In another embodiment, the skin-compatible polymer of the nanodermal gel comprise a polymer selected from a group consisting of sodium polyactylate, alkylacrylate, alkylmethacrylate, acrylic acid, metacrylic acid, polyethylene oxide, polyethylene glycol, polyacrylamide, polyvinyl chloride and combinations thereof, in an amount from 0.01 to 1.0% by weight and preferably in an amount from 0.2 to 0.5% by weight, a natural polymer selected from a group consisting of glucomannan, guar gum, galactomannan, xanthan, gellan, alginate, carrageenan, agar, celluloses, and combinations thereof, in an amount from 0.01 to 5.0% by weight and preferably in an amount from 1.0 to 3.0% by weight, and a polyhydric alcohol selected from glycerin, propylene glycol, butylene glycol, and combinations thereof, in an amount from 5.0 to 20.0% by weight and preferably in an amount from 10.0 to 20.0% by weight.

In a further embodiment, the nanodermal gel further comprises a flavoring agent in an amount from 0.01 to 1.0% by weight and a colorant in an amount from 0.01 to 3.0% by weight.

Examples of the support suitable for use in the present invention, include fluorine-or silicon-treated polymer film, such as polypropylene film, polyethylene terephthalate film, and polyvinylchloride film, woven fabrics, and non-woven fabrics.

In still a further embodiment, the nanowater contains therein a cosmetically active ingredient in an amount from 0.001 to 20.0% by weight based on the total weight of nanowater, said cosmetically active ingredient providing the skin with skin whitening, wrinkle prevention, moisturization, elasticity promotion and/or softening effects and being selected from a group consisting of tocopherol and derivatives thereof retinol and derivatives thereof ascorbic acid and derivatives thereof, cojic acid, hydroquionone and derivatives thereof, hyaluronic acid, ceramides, ceramide analogs, herbal extracts, a green tea extract, caffeine, tea tree oils, plant oils, synthetic oils, marine collagen, vegetable collagen, natural moisturizing factors, ascidian extract, and combinations thereof:

### BRIEF DESCRIPTION OF THE DRAWINGS

The application of the preferred embodiments of the present invention is best understood with reference to the accompanying drawings, in which like reference numerals are used for like and corresponding parts, wherein:

FIG. 1 is a graph of particle size distributions of nanowater;

FIG. 2 is a chromatogram of retinyl palmitate;

FIG. 3 is a plot showing the stability of retinyl palmitate over time; and

FIG. 4 is a plot showing the release capability of nanodermagel according to temperature.

### DESCRIPTION OF THE PREFERRED EMBODIMENTS

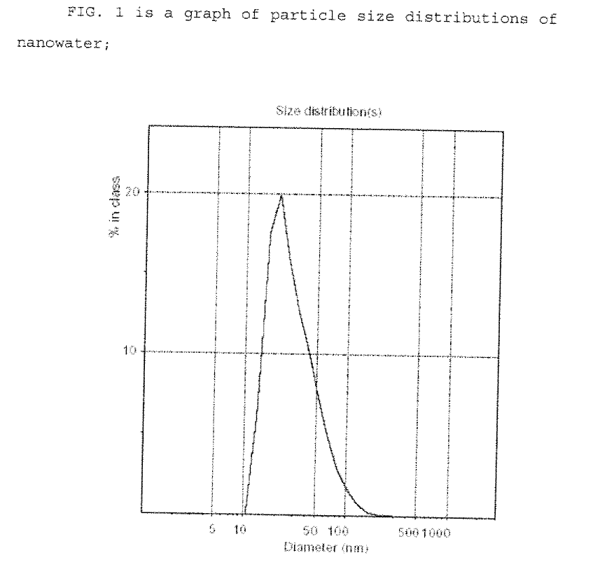

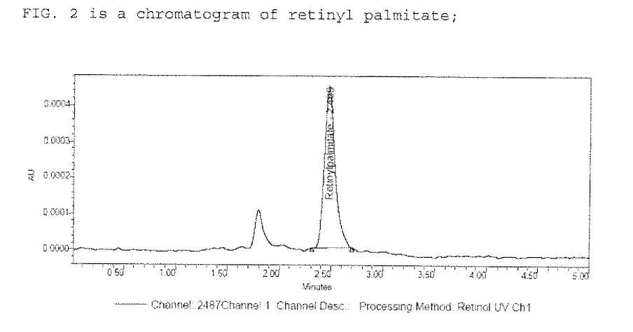

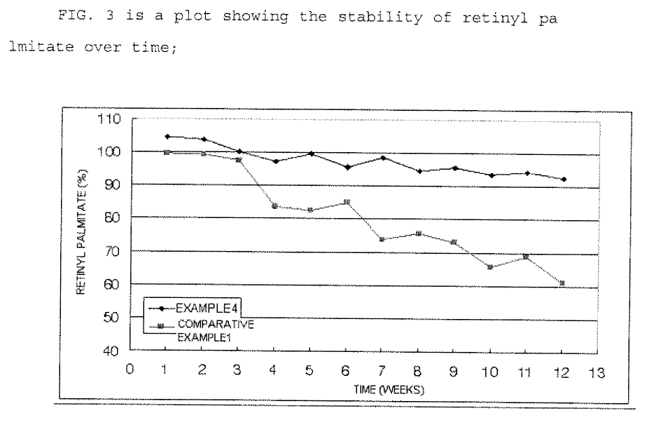

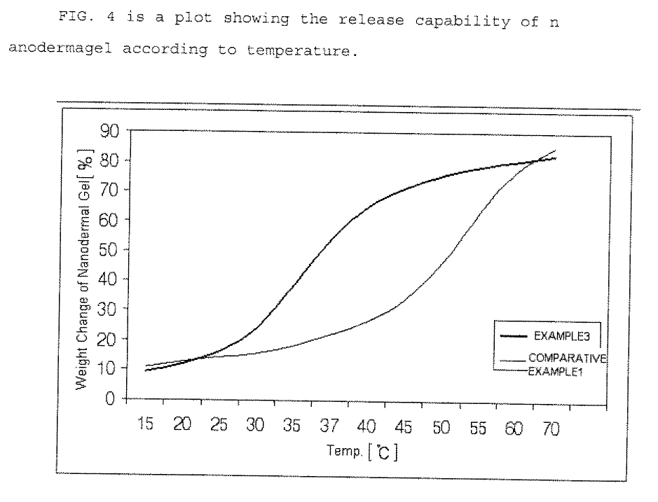

Below, a detailed description is given of the present invention with reference to the accompanying drawings. FIG. 1 is a graph of particle size distributions of nanowater. FIG. 2 is a chromatogram of retinyl palmitate. FIG. 3 is a plot showing the stability of retinyl palmitate over time. FIG. 4 is a plot showing the release capability of nanodermagel with temperature.

A better understanding of the present invention may be given with the following examples which are set forth to illustrate, but are not to be construed to limit the present invention.

### EXAMPLES 1 AND 2

### Production of Nanowater

Nanowater was produced from the compositions listed in table 1, below, as follows.

TABLE 1
Compositions for Nanowater

| Ingredients | EXAMPLE 1 (Wt. %) | EXAMPLE 2 (Wt. %) |
|---|---|---|
| cholesterol | 1.0 | 1.0 |
| Beta-sitosterol | 1.5 | 1.5 |
| Polyhydric alcohols | 7.5 | 7.5 |
| Lecithin | 3.5 | 3.5 |
| Retinyl palmitate | — | 0.01˜20.0 |
| (corresponding to 13.000IU) | | |
| Preservative | Trace | Trace |
| UV Blocking agent | Trace | Trace |
| Purified Water | To 100 | To 100 |

Each of the compositions given in Table 1 was stirred at a predetermined temperature (40˜50° C.) at a speed from 60 to 100 rpm using a paddle mixer, and then at a speed from 2,500 to 3,500 rpm for 2 to 4 min using a homo mixer. The resulting mixture was repetitively treated at a pressure from 700 to 1,000 bar three times in a microfluidizer to produce nanowater having an average particle size from 20 to 100 nm. This nanowater was found to be highly resistant to external factors and to show excellent transdermal delivery and skin affinity. The nanowater prepared according to Example 1 is for general use while the nanowater of Example 2 contains to retinyl palmiate as an active substance in order to be stably delivered into and through the skin. Particle sizes of the nanowater were measured using ZETASIZER 3000HSA (Malvem Instruments Ltd. England) and are shown in FIG. 1.

### EXAMPLES 3 AND 4

### Production of Adhesive Sheet-Type Nanodermal Gel for Transdermal Delivery

Adhesive sheet-type nanodermal gel for transdermal delivery was produced from the components listed in Table 2 below, according to the following processes.

TABLE 2
Nanodermal Gel Compositions For Transdermal Delivery

| Ingredients | EXAMPLE 3 (Wt. %) | EXAMPLE 4 (Wt. %) |
|---|---|---|
| Nanowater | Prepared in Example 1, | Prepared in Example 2 |
| | To 100 | To 100 |
| Agar | 1.0 | 1.0 |
| Glucomannan | 3.0 | 3.0 |
| Sodium polyacrylate | 0.5 | 0.5 |
| Polyhydric alcohols | 20 | 20 |

| | |
|---|---|
| Flavor | Suitable amount |
| Colorant | Suitable amount |

Process 1: Sodium polyacrylate was well dissolved at 80° C. in polyhydric alcohol.

Process 2: Agar and glucomannan was sufficiently dissolved in purified water heated to 90° C.

Process 3: The solutions obtained in Processes 1 and 2 and the nanowater prepared in Example 1 or 2 were homogeneously mixed at 40 to 50° C. by stirring.

Process 4: While a predetermined temperature was maintained, the mixture of Progress 3 was layered to a thickness from 500 to 1,500 82 m on a protection layer of a sheet film using a coating machine to produce a nanowater-containing adhesive sheet-type nanodermal gel for transdermal delivery. Optionally, an embossed film was employed so as to increase the surface area of the resulting application form. In addition, various supplements may be applied according to the purpose of the nanodermal gel.

Process 5 the adhesive sheet-type nanodermal gel was cut into sizes suitable for use on the skin.

### COMPARATIVE EXAMPLES 1 AND 2

### Production of Adhesive Sheet-Type Nanodermal Gel for Transdermal Delivery

The same procedure as in Examples 3 and 4 was repeated except for using the ingredients of Table 3, below, to produce nanodermal gel lacking the nanowater.

TABLE 3
Nanodermal Gel Composition for Transdermal Delivery

| Ingredients | C. Example 1 (Wt %) | C. Example 2 (Wt %) |
|---|---|---|
| Purified Water | To 100 | To 100 |
| Agar | 1.0 | 1.0 |
| Glucomannan | 3.0 | 3.0 |
| Sodium polyacrylate | 0.5 | 0.5 |
| Polyhydric alcohols | 20 | 20 |
| Flavor | Suitable amount | |
| Colorant | Suitable amount | |
| Retinyl palminate | Corresponding to 10,000IU | — |

### EXPERIMENTAL EXAMPLE 1

### Assay of Nanodermal Gels for the Stability of Retinyl Palmiate

The nanodermal gels prepared in Example 4 and Comparative Example 4 were subjected to HPLC analysis to examine the stability of retinyl palmiate according to trial calculation.

Retinyl palmiate was mixed with isopropyl alcohol to afford a series of standard solutions containing 10, 20 and 40 IU of retinyl palmitate. The quantities of retinyl palmitate in the nanodermal gels were determined using the following simple equation obtained from the standard solutions:
Y=aX+b

After being exactly measured, 1 g of the nanodermal gel was added to 50 mL of isopropyl alcohol in a 100 mL volumetric flask. An ultrasonicator was operated for 10 min to completely dissolve the retinyl palminate contained in the nanodermal gel. Thereafter, filtration was conducted using a 0.45 μm filter before HPLC analysis (Water, USA).

The HPLC was equipped with a C18 reverse phase column and conducted using methanol:water 95:5 (v/v) as a mobile phase at a rate of 1.0 mL per min. A UV/Vis detector, also mounted on the HPLC, was used at 325 nm. Results from the HPLC analysis of retinyl palminate stability for 12 weeks are summarized in Table 4, below and shown in the graph of FIG. 3. FIG. 2 is a chromatogram of the retinyl palmitate contained in the nanodermal gel.

TABLE 4
HPLC Analysis for Retinyl Palmitate Stability

| | |
|---|---|
| EXAMPLE4 | Comparative Example 1 |
| EXAMPLE 4 | Comparative Example 1 |

| | Retinyl | | Retinyl | |
|---|---|---|---|---|
| Weeks | Palmitate IU | % | Palmitate IU | % |
| 1 | 10462 | 104.62 | 9958 | 99.58 |
| 2 | 10365 | 103.65 | 9937 | 99.37 |
| 3 | 9997 | 99.97 | 9737 | 97.37 |
| 4 | 9707 | 97.07 | 8350 | 83.5 |
| 5 | 9956 | 99.56 | 8252 | 82.52 |
| 6 | 9567 | 95.67 | 8475 | 84.75 |
| 7 | 9835 | 98.35 | 7375 | 73.75 |
| 8 | 9446 | 94.46 | 7575 | 75.75 |
| 9 | 9547 | 95.47 | 7295 | 72.95 |
| 10 | 9338 | 93.38 | 6575 | 65.75 |
| 11 | 9427 | 94.27 | 6885 | 68.85 |
| 12 | 9243 | 92.43 | 6085 | 60.85 |

As seen in Table 4 and FIG. 3, the nanodermal gel of Example 4, which was prepared from the nanowater containing liposomes of retinyl palmitate, is much more stable than that of Comparative Example 1, which lacks the liposomal retinyl palmitate. These data demonstrate that the nanodermal gel according to the present invention can retain extracts from natural plants and heat-susceptible cosmetic components as well as retinyl palmitate safely and effectively.

### EXPERIMENTAL EXAMPLE 2

### Assay of Nanodermal Gel for Skin Moisturization and Release Capacity

Skin moisturization capacity according to transdermal deliver was compared between the nanodermal gels prepared in Example 3 and Comparative Example 2. In this regard, the skin was assayed for water retention after being treated with the nanodermal gel.

In addition, the nanodermal gel was assayed for release capacity by measuring weight reduction rates according to temperature.

First 40 adults who had worked in the beauty industry divided into two groups of 20 persons, were made to stay for 30 min in an incubation room which was maintained at 22° C. at a relative humidity of 45%, so as to cause their forearms to have a predetermined moisture content. Using Corneometer CM820, the moisture levels of their skin were measured.

After an adhesive sheet (30×40 mm) coated with the nanodermal gel was applied to a predetermined region of the forearm, the skin was measured for change in moisture retention over time. For this, measurements were repeated five times and the average values of the measurement were used to calculate the skin moisturizing capacity according to the following formula, and the results are shown in Table 5.
Skin moisturizing capacity (%)=[(B−A)/A]×100
A: Skin moisture before application of nanodermal gel
B: Skin moisture after application of nanodermal gel

When applied to the skin, the nanodermal gel was evaluated for freshness, water retention, and moisturization according to the following evaluation grades by 10 persons skilled in the beauty art. The results are summarized in Table 5, below.

Evaluation Grades

1: very poor, 2: poor, 3: moderate, 4: good, 5: very good

TABLE 5
Skin Moisturizing Capacity and Feeling of Nanodermal Gel

| | Example 3 | C. Example 2 |
|---|---|---|
| Skin Moisture | 91.08 | 47.40 |
| Freshness | 4.17 | 3.86 |
| Water retention | 4.69 | 3.35 |
| Moisturization | 4.95 | 3.16 |

As apparent from the data of Table 5, the adhesive sheet type nanodermal gel containing the nanowater of Example 3 has excellent skin moisturizing capacity. In addition, the adhesive sheet type nanodermal gel according to the present invention is superior to the nanodermal gel containing water with respect to freshness, moisture retention and moisturization.

After being tightly packed with aluminum foil, the product forms of nanodermal gel prepared in Example 3 and Comparative Example 2 were allowed to stand for 20 min at predetermined temperatures and opened to remove the liquid leached therefrom with paper tissue. The nanodermal gel was then weighed. The weight reduction of the nanodermal gel was expressed as percentages relative to the initial weight and is shown in Table 6, below. In FIG. 4, the release capacity of the nanodermal gel is plotted against temperature.

TABLE 6
Weight Reduction Change of Nanodermal Gel with Temperature

Reduction (%)

| Temp. (° C.) | Example 3 | C. Example 2 |
|---|---|---|
| 15 | 9.3 | 10.9 |
| 20 | 12 | 12.9 |
| 30 | 24 | 15.6 |
| 35 | 39 | 18.3 |
| 40 | 54.5 | 22.2 |
| 45 | 65.5 | 26.9 |
| 50 | 71.7 | 34.3 |
| 55 | 76 | 47.1 |
| 60 | 78.7 | 64.3 |
| 65 | 80.5 | 77.5 |
| 70 | 82.5 | 85.3 |

As seen in Table 6, the nanowater-containing nanodermal gel according to Example 3 had sharply increased release capacity at around the body temperature while the release capacity of the nanodermal gel of Comparative Example 2 was gently increased over the entire temperature range. The nanodermal gel of the present invention is found to actively release its contents at around the body temperature. Therefore, when applied to the skin, the nanodermal gel of the present invention reacts at the temperature of the skin to allow the liposomal nanowater to be released effectively. That is, the nanodermal gel of the present invention is found to have optimal release conditions for transdermal delivery.

As described hereinbefore, the patch-type nanodermal gel including liposomal nanowater according to the present invention can deliver cosmetically active substances, such as those functioning to moisturize the skin, into and through the skin stably and effectively. It can he also effectively prepared according to the method of the present invention.

Examples are described in terms of the preferred embodiment of present invention. However, it should be understood that this disclosure is not limited to the explicit description of the present invention. The description and the claims of the present invention are to be interpreted as covering all variations and modifications that fall within the true scope of this invention.

What is claimed is:

1. A patch-type cosmetic composition for transdermal delivery, based on nanodermal gel, comprising:
nanowater in an amount from 0.01 to 80.0% by weight;
a skin-compatible polymer in an amount from 0.01 to 10.0% by weight; and
a polyhydric alcohol in an amount from 5.0 to 20.0% by weight, based on the total weight of the nanodermal gel.

2. A method for preparing a patch-type cosmetic, comprising:
uniformly dissolving a skin-compatible polymeric substance in purified water and a polyhydric alcohol at a warm temperature;
homogeneously mixing the solution with nanowater at 40 to 50° C., along with a flavor and a colorant, by stirring to produce nanodermal gel including the nanowater therein;
layering nanodermal gel to a thickness from 500 to 1,500 μm on a film; and
cutting the film to a predetermined size for patching.

3. The method according to claim 2, wherein the film has an embossed surface and is reinforced by a support.

4. The method according to claim 2, wherein the nanowater ranges in size from 20 to 100 nm and is prepared by mixing 2.0˜4.0 wt % of lecithin, 2.0˜5.0 wt % of cholesterol and 1.0˜2.5 wt % of beta-sitosterol based on the total weight of the nanowater, at 40˜50° C. by gently stirring at 60˜100 rpm using a paddle mixer and vigorously stirring at 2,500˜3,500 rpm using a homo mixer for 2 to 4 min, and then by passing the mixture at 700˜1,000 box three times through a microfluidizer.

5. The method according to claim 3, wherein the nanowater contains therein a cosmetically active ingredient in an amount from 0.001 to 20.0% by weight based on the total weight of nanowater, said cosmetically active ingredient providing the skin with skin whitening, wrinkle prevention, moisturization, elasticity enhancement and/or softening effects and being selected from a group consisting of tocopherol and derivatives thereof, retinol and derivatives thereof, ascorbic acid and derivatives thereof, cojic acid, hydroquinone and derivatives thereof, hyaluronic acid, ceramides, ceramide analogs, herbal extracts, a green tea extract, caffeine, tea tree oils, plant oils, synthetic oils, marine collagen, vegetable collagen, natural moisturizing factors, ascidian extract, and combinations thereof.
